(19) United States
(12) Patent Application Publication
(10) Pub. No.: US 2009/0069549 A1
(43) Pub. Date: Mar. 12, 2009

(54) Protein Extraction buffer, a kit comprising it and method of its use

(76) Inventors: Elias Georges, Laval (CA); Corinne Benquet, Laval (CA); Julie Lantheir, Laval (CA)

Correspondence Address: WILMERHALE/BOSTON, 60 STATE STREET, BOSTON, MA, 02109, US

(21) Appl. No.: 11/900,671

(22) Filed: Sep. 12, 2007

(51) Int. Cl. C07K 1/14; C09K 3/00

(52) U.S. Cl. 530/412; 252/183.12

(57) ABSTRACT

Disclosed are detergent-free protein extract buffers to extract proteins from formalin-fixed paraffin-embedded tissue sections, kit comprising the buffer, and methods for using the buffer to extract proteins from formalin-fixed, paraffin-embedded tissue sections.

### FIELD OF THE INVENTION

The present invention relates generally to the field of proteomics. More specifically, the invention relates to protein extraction from formalin-fixed paraffin-embedded (“FFPE”) tissue sections.

### BACKGROUND OF THE INVENTION

The application of proteomics-oriented technology in cell biology and medical research has expanded greatly in recent years. In any proteomics study, it is accepted that the most critical step is sample extraction and preparation. The problem is especially difficult with respect to proteins extracted from FFPE tissue sections. Formalin fixation, generally followed by paraffin embedding, is the standard and well-established processing method employed by pathologists. This treatment conserves and stabilizes biopsy samples for years. The vast archive of pathologically characterized samples that exists worldwide allows for the proteomic analysis of FFPE tissues and retrospective biomarker investigations.

Ideally, the proteins extracted from FFPE should have their antibody epitopes preserved to allow for subsequent analysis of these protein samples in applications such as SDS-PAGE, Western blot analysis, immunoassay procedures such as ELISA and tryptic digestion/mass spectrometry. Presently existing protein extraction buffers are not able to accomplish the preservation of antibody epitopes of protein samples extracted from FFPE tissue sections sufficiently well.

Thus, there remains a need for an extraction buffer to extract proteins from FFPE tissue sections that preserves the antibody epitopes of the extracted proteins well, allowing best antibody detection of extracted proteins.

### SUMMARY OF THE INVENTION

It has been determined that a protein extraction buffer which comprises Tris-HCl, guanidine hydrochloride (GnHCl), dithiothreitol (DTT) and a protease inhibitor is best at extracting proteins from FFPE tissue sections so as to preserve the antibody epitopes of the extracted proteins best. This determination has been exploited to provide the present invention which related to extraction of proteins from Paraffin slides.

In one aspect, the invention provides a detergent-free protein extraction buffer which comprises Tris-HCl, GnHCl, DTT and a protease inhibitor. In specific embodiments, the extraction buffer comprises: 10 mM to 250 mM Tris-HCl, pH 7.0 to pH 10; 2 M to 8 M GnHCl; 10 mM to 200 mM DTT; and a protease inhibitor. In certain embodiments, the protein extraction buffer comprises: 10 mM to 80 mM Tris-HCl, pH 8.0 to pH 8.6; 5 M to 8 M GnHCl; 50 mM to 110 mM DTT; and a protease inhibitor. In some other embodiments, the protein extraction buffer comprises: 20 mM Tris-HCl, pH 8.4; 6 M GnHCl; 100 mM DTT; and a protease inhibitor. In one embodiment, the protein extraction buffer consists essentially of 20 mM Tris-HCl, pH 8.4; 6 M GnHCl; 100 mM DTT; and a protease inhibitor. In certain embodiments, the protease inhibitor is one or more of the following protease inhibitors: Leupeptin, AEBSF, Aprotinin, Pepstatin A, E-64, and EDTA. In one specific embodiment, the protease inhibitor is 1 μM to 100 μM (10 μM) Leupeptin. In another specific embodiment, the protease inhibitor is 10 μM to 1 mM (100 μM) AEBSF. In another specific embodiment, the protease inhibitor is 0.1 μM to 10 μM (0.3 μM) Aprotinin. In another specific embodiment, the protease inhibitor is 0.1 μM to 10 μM (1 μM) Pepstatin A. In another specific embodiment, the protease inhibitor is 0.1 μM to 10 μM (1 μM) E-64. In yet another specific embodiment, the protease inhibitor is 0.1 mM to 10 mM (1 mM) EDTA.

In another aspect, this invention provides a method for extracting protein from a formalin-fixed, paraffin-embedded tissue section. The method comprises obtaining a deparaffinized, formalin-fixed, paraffin-embedded tissue section; incubating the tissue section with a detergent-free protein extraction buffer according to the invention; wherein the extraction buffer significantly preserves protein epitopes for antibody recognition of an extracted protein; and recovering from the contacted tissue section a soluble fraction containing one or more proteins. In certain embodiments, the tissue section is incubated in the protein extraction buffer at two different temperatures. In certain embodiments, the two different temperatures are about 100° C. and about 60° C. In yet other embodiment, the tissue section is first incubated in the protein extraction buffer at about 100° C. for about twenty minutes and then at about 60° C. for about two hours.

In another aspect, the present invention provides a kit for extracting protein from a deparaffinized, formalin-fixed, paraffin-embedded tissue section. The kit comprises a detergent-free protein extraction buffer according to the invention, wherein the extraction buffer significantly preserves protein epitopes for antibody recognition on the extracted protein; and a manual for use.

In yet another aspect, the present invention provides a kit for extracting protein from a deparaffinized, formalin-fixed, paraffin-embedded tissue section. The kit comprises 10 mM to 250 mM Tris-HCl, pH 7.0-pH 10; 2 M to 8 M GnHCl; 10 mM to 200 mM DTT, and a protease inhibitor, the Tris, GnHCl, DTT and protease inhibitor being components of a protein extraction buffer, and a manual for use and for directing the mixing of the individual components of the protein extraction buffer. In certain embodiments, the kit comprises 10 mM to 80 mM Tris-HCl, pH 8.0 to pH 8.6; 5 M to 8 M GnHCl; 50 mM to 110 mM DTT; and a protease inhibitor. In certain other embodiments, the kit comprises: 20 mM Tris-HCl, pH 8.4; 6 M GnHCl; 100 mM DTT; and a protease inhibitor. In one embodiment, the kit consists essentially of: 10 mM to 80 mM Tris-HCl, pH 8.0 to 8.6; 5 M to 8 M GnHCl; 50 mM to 110 mM DTT; and a protease inhibitor. In certain embodiments, the protease inhibitor is one or more of the following protease inhibitors: Leupeptin, AEBSF, Aprotinin, Pepstatin A, E-64 and EDTA. In one specific embodiment, the protease inhibitor is 1 μM to 100 μM (10 μM) Leupeptin. In another specific embodiment, the protease inhibitor is 10 μM to 1 mM (100 μM) AEBSF. In another specific embodiment, the protease inhibitor is 0.1 μM to 10 μM (0.3 μM) Aprotinin. In another specific embodiment, the protease inhibitor is 0.1 μM to 10 μM (1 μM) Pepstatin A. In another specific embodiment, the protease inhibitor is 0.1 μM to 10 μM (1 μM) E-64. In yet another specific embodiment, the protease inhibitor is 0.1 mM to 10 mM (1 mM) EDTA.

### BRIEF DESCRIPTION OF THE FIGURES

The foregoing and other objects of the present invention, the various features thereof, as well as the invention itself may be more fully understood from the following description, when read together with the accompanying drawings in which:

FIG. 1 is a diagrammatic representation of a generalized protocol for extracting proteins from FFPE tissue sections and subsequently analyzing the extracted proteins.

FIG. 2 is a photographic representation of a Western blot of FFPE breast tumor tissue extracted by a method of the invention. The 4 lanes are, from left to right, proliferating nuclear antigen (PCNA), β-actin, cytokeratin 19 and E-cadherin. Molecular weight markers are shown on the right.

FIG. 3 is a graphic representation of the results of ELISA of proteins extracted by three different extraction buffers: Epitex™ FFPE buffer (a buffer of the invention) and extraction buffers from two commercial suppliers—KPL, Inc. (Gaithersburg, Md. USA) for the ProteoSOL Tissue Extraction System and QIAGEN (Valencia, Calif. USA) for the Qproteome FFPE Tissue Kit. The Y axis displays antigen detected as OD₄₅₀.

FIG. 4 is a diagrammatic representation of a general procedure to extract proteins from FFPE tissue sections.

FIG. 5 is a graphic representation of the results of ELISA of proteins extracted by three different extraction buffers: ABP-FFPE buffer (a buffer of the invention) and extraction buffers from two commercial suppliers—the ProteoSOL Tissue Extraction System of KPL, Inc. (Gaithersburg, Md. USA) and the Qproteome FFPE Tissue Kit of QIAGEN (Valencia, Calif. USA). The Y axis displays antigen detected as OD₄₅₀.

FIG. 6 is a graphic representation of the results of ELISA of proteins extracted by three different extraction buffers: ABP-FFPE buffer (a buffer of the invention) and extraction buffers from two commercial suppliers, the ProteoSOL Tissue Extraction System of KPL, Inc. (Gaithersburg, Md. USA) and the Qproteome FFPE Tissue Kit of QIAGEN (Valencia, Calif. USA). The Y axis displays antigen detected as OD₄₅₀.

FIG. 7 is a photographic representation of a Western blot of FFPE breast tumor tissue extracted by a method of the invention. The 5 lanes are, from left to right, cellular retinoic-acid binding protein II (CRAB-PII), proliferating cell nuclear antigen (PCNA), β-actin, cytokeratin 19 and E-cadherin. Molecular weight markers are shown on the right.

FIG. 8 is a graphic representation of three ELISA experiments of three proteins, extracted in three separate extractions from FFPE sections using an extraction buffer of the invention.

FIG. 9 is a graphic representation of comparison study of the ability of five different extraction buffers (Tris/urea, Tris/guanidine, Tris/guanidine/DTT, ProteoSOL and Qproteome) to extract proteins from breast tumor biomarkers from FFPE tissue samples and to preserve antibody epitopes of five different proteins for ELISA.

### DETAILED DESCRIPTION OF THE INVENTION

Patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

### 1.1. General

The present invention provides, in part, detergent-free extraction buffers and kits for extracting proteins and polypeptides, ranging from full-length proteins to small polypeptides, from formalin-fixed paraffin embedded (“FFPE”) tissue sections. The present invention also provides, in part, methods for extracting proteins using the detergent-free extraction buffers provided by this invention. Following extraction, the extracted protein samples can be used, e.g., in downstream proteomic analysis and application, including, but not limited to, SDS-PAGE, Western blot analysis, immunoassays, such as ELISA, and tryptic digestion/mass spectrometry. Because the antibody epitopes of the extracted proteins are well preserved, the extracted protein samples are also useful in applications in which an antibody to one of the extracted protein or polypeptide is used.

### 1.2. Detergent-Free Protein Extraction Buffer

The invention provides, in part, a detergent-free protein extraction buffer, which comprises Tris-HCl, GnHCl, DTT, and a protease inhibitor.

The concentration of Tris-HCl in the protein extraction buffer can be between about 10 mM and about 250 mM or between about 10 mM to about 80 mM; one useful concentration of Tris-HCl is 20 mM. Tris base can be obtained from any commercial source such a Sigma-Aldrich (St. Louis, Mo., USA) (Trizma®). The pH of the Tris-HCl component can be between pH 7.0 to pH 10.0 or between pH 8.0 to pH 8.6. One useful pH for the Tris-HCl is pH 8.4. pH can be adjusted using HCl.

The GnHCl concentration of the protein extraction buffer can be between 2 M and 8 M or between 5 M to 8 M. One useful GnHCl concentration is 6 M. GnHCl can be obtained from any commercial source, such as Sigma-Aldrich (St. Louis, Mo., USA).

DTT is present in the extraction buffer at a concentration of about 10 mM to about 200 mM, or about 50 mM to about 110 mM. One useful DTT concentration is 100 mM. DTT can be obtained from any commercial source, such as Sigma-Aldrich (St. Louis, Mo., USA).

As used herein, “about” means a numeric value having a range of ±10% around the cited value.

The protease inhibitor of the extract buffer can be any one or more of the protease inhibitors well known in the art and added to the extraction buffer at a concentration effective for inhibiting proteases. The protease inhibitor can be, for example, one or more of the following protease inhibitors or combination thereof: Leupeptin, AEBSF, Aprotinin, Pepstatin A, E-64 and EDTA. Useful non-limiting concentrations of any of these protease inhibitors are: 10 μM Leupeptin, 100 μM AEBSF, 0.3 μM Aprotinin, 1 μM Pepstatin A, 1 μM E-64, and 1 mM EDTA. These protease inhibitors can also be used in other concentrations that are effective for inhibiting protease activity. The protease inhibitors can be obtained from commercial sources, such as Sigma-Aldrich, St. Louis Mo.

Simple general chemistry is used to titrate the pH of the Tris-HCl to the desired pH, and to add each ingredient to the desired concentration.

The detergent-free extraction buffer of the invention can be used to extract proteins from, e.g., FFPE tissue sections. The term “proteins” used in the context of proteins extracted from FFPE tissue section means any protein which is full-length, a polypeptide, a fragment of full-length protein, as a small polypeptide. This extraction buffer is useful for extracting proteins from FFPE tissue culture to preserve antibody epitopes on the extracted proteins.

Once extracted, the proteins can be used, e.g., in proteomic applications. The extracted proteins may be subjected to SDS-PAGE, Western blot, immunoassays such as ELISA and tryptic digestion/mass spectrometry, or may be used in other biochemical analysis and proteomic biomarker discovery methods and procedures that make use of extracted proteins.

### 1.3. A Method for Extracting Proteins From FFPE Tissue Sections

This invention also provides, in part, a method for extracting protein from a formalin-fixed, paraffin-embedded tissue section, comprising the steps of: (a) obtaining a deparaffinized, formalin-fixed, paraffin-embedded tissue section; (b) incubating the tissue section with a detergent-free protein extraction buffer according to the invention (see, e.g., the preceding section), wherein the extraction buffer significantly preserves protein epitopes for antibody recognition of an extracted protein; and (c) recovering from the contacted tissue section a soluble fraction containing one or more proteins.

The protein extraction buffer used in the method of the invention is optimized for extracting total proteins from, e.g., FFPE tissue sections. Formalin fixation, generally followed by paraffin embedding, is the standard and well-established processing method employed by pathology laboratories for tissue samples. This treatment conserves and stabilizes biopsy samples for years, providing access to multiple samples from a long period of time. The tissue sections can be 10 mm² and 10 μm in thickness. Sample size can be reduced considerably if the detection method is very sensitive. Moreover, sample size also depends on the abundance of the proteins or peptides in question and the nature of the material extracted (e.g. fatty tissue may require greater sample size).

The FFPE tissue sections can be obtained from a number of sources, including pathology laboratories. For example, the FFPE tissue sections can be sections from patients suffering any disease or condition known, including, but not limited to, cancer, diabetes, heart diseases, neurodegenerative disease such as Alzheimer's and Parkinson's diseases, multiple sclerosis, baldness, obesity, etc.

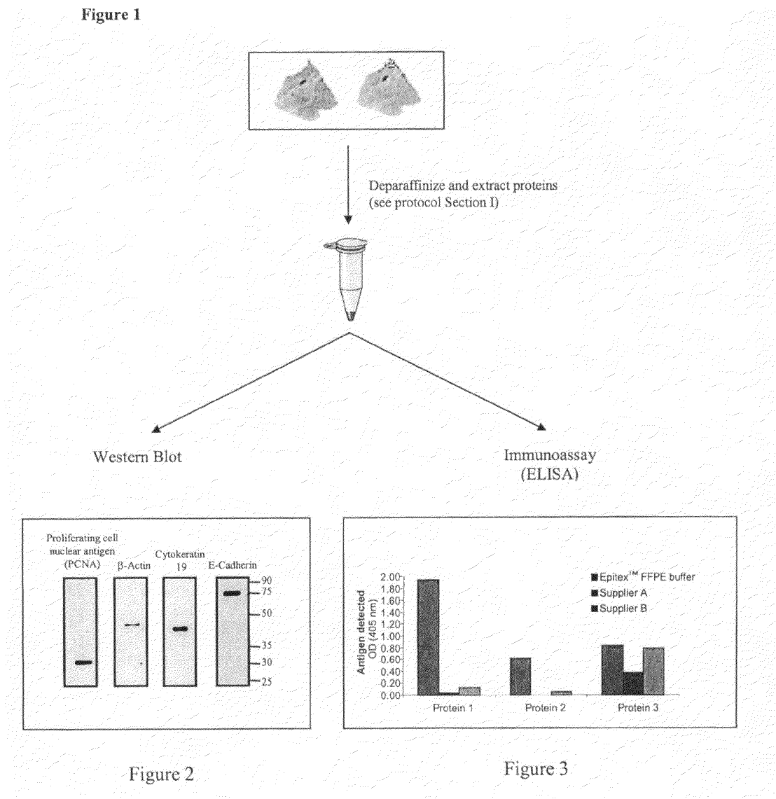

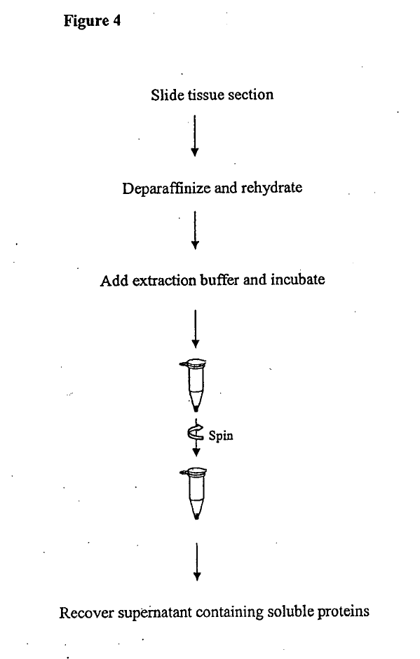

The FFPE sections are first deparaffinized using any method known in the art. For example, the sections can be deparaffinized by xylene followed by ethanol, as described in Examples 1-3, below. All the steps for deparaffinization can be done at room temperature. The cleaned tissues are then incubated in the extraction buffer according to the invention, which contains protease inhibitors, and incubated. Two different temperatures can be used for incubation to allow antigen retrieval and untangling of protein. For example, two useful temperatures that can be used are about 100° C. and about 60° C. The tissue section can be first incubated in the protein extraction buffer at about 100° C. for, e.g., about twenty minutes, and then at about 60° C. for, e.g., about two hours. This last step (incubation in the protein extraction buffer at about 60° C. for, e.g., about two hours) allows preservation of protein epitopes for antibody recognition. After centrifugation, the soluble proteins are recovered and can be used for, e.g., Western blot, immunoassay analyses or further digested and analyzed by mass spectrometry, and any other applications requiring extracted proteins that have their antibody epitopes well preserved. FIGS. 1 and 4 provide schematic diagrams of examples of extraction procedures. Also see Examples 1-3. The extraction buffer of the invention preserves antibody epitopes of extracted proteins from FFPE tissue sections (See, e.g., Examples 1-3 and Figures cited therein).

Example 4 shows that the buffer provided by this invention can identify biomarkers from malignant FFPE tissue sections such as breast tumor samples. These biomarkers are shown to be elevated in breast cancer samples as compared to benign samples.

### 1.4. Kits

Aspects of the invention additionally provide kits for extracting proteins from FFPE. The kits may be marketed as Epitex™ kits. The kit may provide components useful for extracting protein from a deparaffinized, formalin-fixed, paraffin-embedded tissue section. The kit may comprise: (a) the detergent-free protein extraction buffer of this invention (see, e.g., section 1.2 of Detailed Description of the Invention), wherein the extraction buffer significantly preserves protein epitopes for antibody recognition of an extracted protein; and (b) a manual for use.

In another aspect, this invention provides a kit for extracting protein from a deparaffinized formalin-fixed paraffin-embedded tissue section. The kit comprises Tris-HCl, GnHCl, DTT, and a protease inhibitor. For example, the kit may comprise: (a) 10 mM to 250 mM Tris-HCl, pH 7.0 to pH 10; (b) 2 M to 8M GnHCl; (c) 10 mM to 200 mM DTT; and (d) a protease inhibitor, components (a)-(d) being components of a protein extraction buffer of the invention; and (e) a manual for use and for directing the mixing of the individual components of the protein extraction buffer and/or for its use. This kit can comprise or consist essentially of the following components: (a) 10 mM to 80 mM Tris-HCl, pH 8.0 to pH 8.6; (b) 5M to 8M GnHCl; (c) 50 mM to 110 mM DTT; and (d) a protease inhibitor. This kit can also comprise or consist essentially of: (a) 20 mM Tris-HCl, pH 8.4; (b) 6M GnHCl; (c) 100 mM DTT; and (d) a protease inhibitor. The protease inhibitor can be any one or more of the protease inhibitors well known in the art. Nonlimiting useful protease inhibitors include Leupeptin, AEBSF, Aprotinin, Pepstatin A, E-64, and EDTA. In certain embodiments, the protease inhibitor is one or more of the following protease inhibitors. For example, the inhibitor may be 10 μM Leupeptin, 100 μM AEBSF, 0.3 μM Aprotinin, 1 μM Pepstatin A, 1 μM E-64 and 1 mM EDTA.

These kits can be stored at −20° C. for up to six months.

The kits may also have a manual accompanying them to explain, among other things, conditions for storage and methods of reconstitution, provide exemplary protocols for use, and provide safety warnings, if any.

Depending on the FFPE tissue sections used, a kit can be used to identify biomarkers from any disease or condition. For example, if the FFPE tissue sections are from breast cancer patients, the kit can be used to identify breast cancer biomarkers, by, for example, ELISA.

### EXAMPLES

Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Deparaffinization and Protein Extraction from FFPE Tissue Sections

FFPE slides are obtained from any source, such as a pathology lab. Section from FFPE slides are up to about 10 μm thick. Up to 2 sections, each with a thickness of about 10 μm and area of up to about 100 mm², are combined in one preparation. Smaller sections are also combined for one preparation. The yield of extracted protein depends on the amount and the nature of the starting material and may vary depending on cell type.

### A. Deparaffinization

Before deparaffinization, slides are kept at room temperature for 60 min. or heated at 60° C. for 20 min. in a horizontal position. The slide is immersed in xylene for 10 min. This step is repeated once in new xylene solution for 10 min. The slide is then immersed in 100% ethanol for 5 min.; followed by another immersion in 85% ethanol for 1 min.; followed by yet another immersion in 70% ethanol for 1 min. The slide is then immersed in high purity water for 1 min. Excess water is removed from the slide, but sections are not allowed to dry out. The sections are then transferred to a clean Eppendorf tube using a razor blade.

### B. Protein Extraction

35 μl Extraction Buffer is pipetted into one Eppendorf tube containing the tissue section(s). The sample in the Eppendorf tube is then vortexed for 5 sec. The sample in the Eppendorf tube is incubated on a heating block at 100° C. for 20 min. The sample in the Eppendorf tube is centrifuged for 1 min. at 1000×g. The sample is then vortexed for 5 sec. and is incubated at 60° C. for 2 hours. Following the incubation, the sample is centrifuged for 10 min. at 15 000×g at 4° C. The supernatant (soluble fraction) is transferred to a new 1.5 ml Eppendorf tube.

The protein yield is quantified using the Bradford Reagent (Sigma-Aldrich, St. Louis, Mo. USA).

Extraction steps are performed in siliconized tubes in order to prevent protein adsorption to the plastic and limit protein loss. After quantification, extracted proteins are stored for up to 1 week at 4° C. For longer-term storage, the proteins aliquots are stored at −20° C.

Using this protocol, the expected protein yield is about 10 μg-30 μg protein from FFPE slides using a tissue size of 10×10 mm and 2×10 μm thick. The yield of extracted protein depends on the amount and the nature of the starting material and may vary depending on cell type.

### C. SDS-PAGE and Western Blot Analysis

The sample is precipitated prior to loading on SDS-PAGE and Western Blot analysis in order to remove any interfering agents. Acetone is useful for protein precipitation. Other reagents well known in the art used to precipitate protein may also be used.

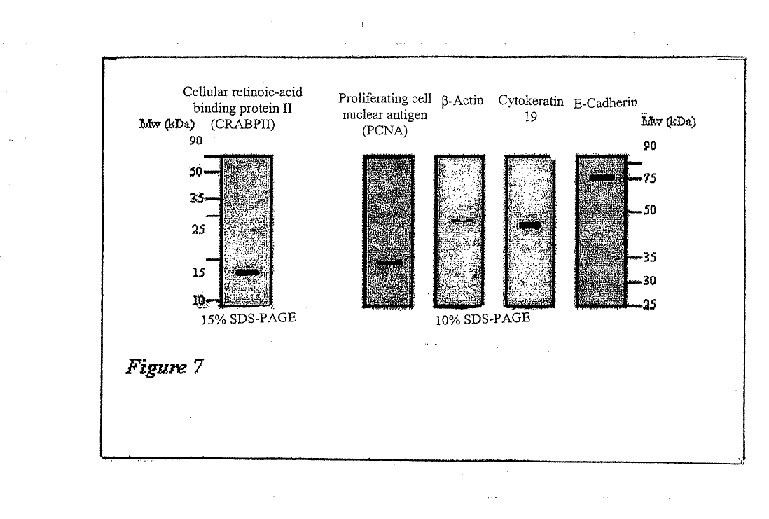

FIGS. 2 and 7 demonstrate that the extraction buffer and method or the invention allow for clear signals of several biomarkers on Western blot analysis, showing specificity. In FIG. 2, FFPE breast tumor tissue was extracted by the method of the invention and the extracted proteins were precipitated with acetone. The pellet was resuspended in SDS sample buffer, separated by SDS-PAGE and transferred to nitrocellulose membrane.

Transferred proteins were then probed with specific antibodies: antibodies to PCNA, β-actin, cytokeratin 19 and E-cadherin (FIG. 2). The results show that each of the specific antibodies reacted with one protein of the appropriate size in each experiment. In FIG. 7, breast tumor FFPE tissue sections were extracted by a method of the invention and the extracted proteins precipitated. The pellets were suspended in SDS sample buffer, separated by SDS-PAGE and transferred to nitrocellulose membrane. Transferred proteins were then probed with the specified antibodies: antibodies to CRAB-PII, PCNA, β-actin, cytokeratin 19, and E-cadherin.

FIG. 3 is a comparative study of the ability of proteins extracted by three different buffers, one being of this invention (the Epitex™ FFPE buffer), one being the ProteoSOL Tissue Extraction System from KPL, Inc. and one being the Qproteome FFPE Tissue Kit from QIAGEN, to preserve antibody epitopes after extraction for subsequent analysis by ELISA. The quantity of proteins reactive with a specific antibody was quantified spectrophotmetrically and is shown as OD₄₅₀. The results of FIG. 3 show that the extraction buffer of the invention (Epitex™) is superior to two commercially available extraction buffers in ELISA, showing excellent antibody epitope preservation for three biomarkers (Proteins 1, 2, and 3).

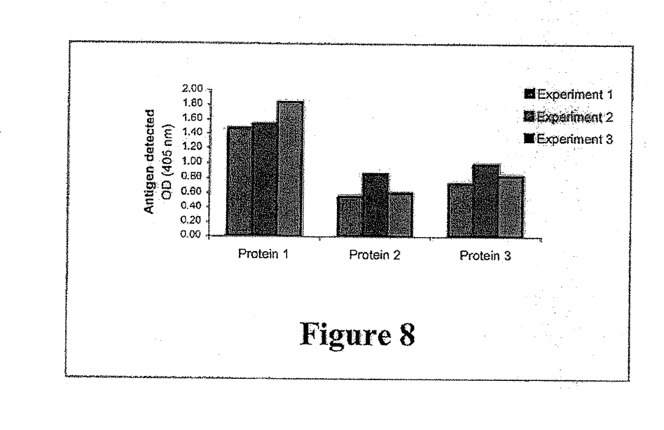

FIG. 8 shows the reproducibility of this protocol for three biomarkers (Proteins 1, 2, and 3). Three breast tumor FFPE sections (10 μm each) were processed by a method of the invention. Expression of three biomarkers was measured by ELISA in these three samples. The amount of each protein is quantitated spectrophotmetrically after ELISA.

### Example 2

### Comparison of Proteins Obtained From Breast Tumor Samples Using Different Extraction Buffers

### A. Deparaffinization of Tissue Sections

For each extraction condition, 2 slides of the same breast tumor patient (HuCAT295, USBiomax, Rockville Md.) were used. To deparaffinize FFPE tissue sections, slides were immersed in xylene for 5 min.; in fresh xylene for 5 min.; in 100% ethanol for 5 min.; in 85% ethanol for 1 min.; in 70% ethanol for 1 min.; and in high purity water for 1 min. Excess water was removed and tissue sections were transferred to a clean Eppendorf tube using a razor blade by pooling 2 tissue sections together.

### B. Protein Extraction

35 μl ABP extraction buffer (of the invention) was added to the tissue section. The tissue was broken using a plastic pestle and heated at 100° C. for 20 min. After a quick spin down, the tissue was broken again with the plastic pestle and incubated at 60° C. for 2 hours. The sample was then centrifuged at 15,000 g for 10 min. at 4° C. and the soluble fraction was recovered.

The KPL Tissue Extraction System was used following manufacturer's recommendations (KPL, Inc., Gaithersburg, Md., USA). KPL's tissue extraction system is the proteoSOL Extraction System. Specifically, 50 μl buffer A was added to the tissue section and heated at 95° C. for 90 min. Every 20 min., the sample was mixed by vortexing and quickly spun to remove the condensation in the cap. The sample was then cooled on ice for 2 min.; then buffer B was added (2.5 μl) and incubated at 37° C. for 1 hour. Every 20 min., the sample was mixed by vortexing and quickly spun to remove the condensation in the cap. The sample was then reheated at 95° C. for 5 min. and centrifuged at 15,000 g for 10 min. at 4° C. The soluble fraction was then recovered.

The QIAGEN Tissue Extraction System was used following manufacturer's recommendations (QIAGEN, Valencia Calif., USA). QIAGEN's tissue extraction system is the QProteome FFPE Tissue Kit. Specifically, 100 μl extraction buffer was added to the tissue section and heated at 100° C. for 20 min. After a quick spin down to remove the condensation in the cap, the sample was incubated at 80° C. for 2 hours. Every 20 min., the sample was mixed by vortexing and quickly spun to remove the condensation in the cap. The sample was then centrifuged at 15,000 g for 10 min. at 4° C. and the soluble fraction was recovered.

The total amount of proteins extracted with each method was then assayed. For ABP-FFPE and KPL's methods, the Bradford Reagent (Sigma-Aldrich, St. Louis, Mo.) was used following manufacturer's protocol. For QIAGEN's method, the DC Protein Assay (Bio-Rad, Hercules, Calif.) was used since the detergents present in the extraction buffer interacted with the Bradford reagent.

### C. Conclusion

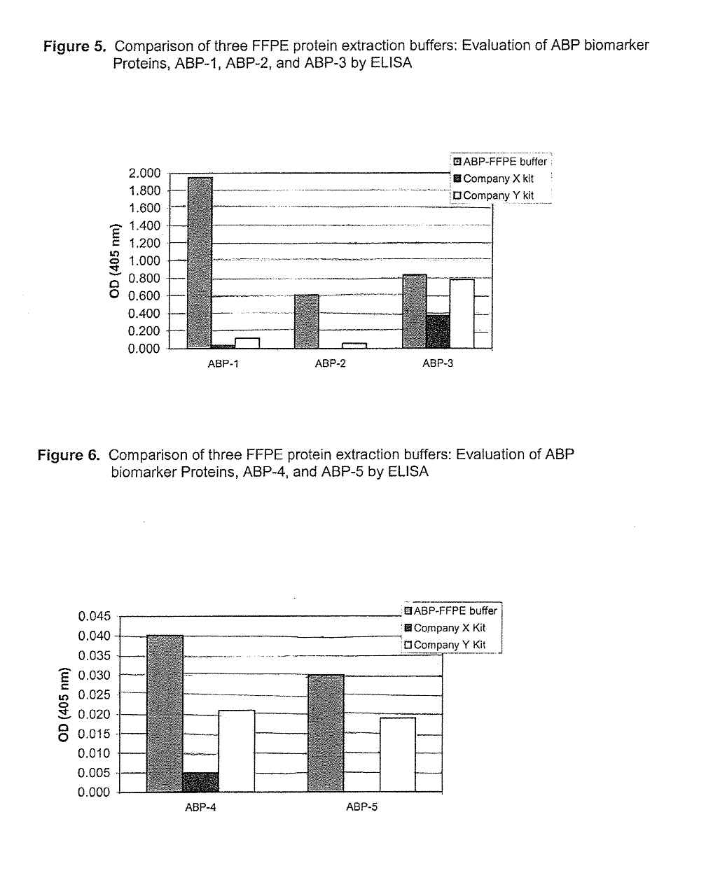

As can be seen in FIGS. 5 and 6, the ABP extraction buffer preserves antibody epitopes and detectable signal of a number of biomarkers in an ELISA. FIGS. 5 and 6 are graphic representation of experiments to evaluate the preservation of antibody epitopes in three biomarkers extracted from breast tumor FFPE tissue sections using three different buffers: ABP-FFPE (a buffer of this invention) and buffers from KPL, Inc. and QIAGEN.

### Example 3

### Comparison of Extraction Buffers for FFPE

### A. Extraction Buffer of the Invention

Two 10 μm tissue section slides were taken from the same patient. The slides were immersed in xylene for 5 min. and immersed again in fresh xylene solution for 5 min. The slides were then immersed in 100% ethanol for 5 min. This step was repeated. The slides were then immersed in 85% ethanol for 1 min., followed by immersion in 70% ethanol for 1 min. The slides were then immersed in water for 1 min. Excess water was removed from the slides and the sections are transferred to a clean Eppendorf tube using a razor blade.

35 μl Extraction Buffer was added to one Eppendorf tube containing the tissue section. The sample tube was heated at 100° C. for 20 min. The sample tube was centrifuged to spin down the liquid. The sample tube was then incubated at 60° C. for 2 hours, and vortexed every 30 min. during the incubation. The sample tube was then centrifuged for 10 min. at 15,000×g at 4° C. The soluble fraction was recovered.

### B. ProteoSOL Tissue Extraction System (KPL, Inc., Gaithersburg Md., USA)

Two 10 μm tissue section slides from the same patient were used. The slides were immersed in xylene for 5 min.; this step was repeated once in new xylene solution for 5 min. The slides were immersed in 100% ethanol for 5 min. This step is repeated once. The slides were immersed in 85% ethanol for 1 min. The slides were immersed in 70% ethanol for 1 min. The slides were immersed in water for 1 min. Excess water was removed and the sections were transferred to a clean Eppendorf tube using a razor blade.

50 μl of buffer A from the manufacturer was added to one Eppendorf tube containing the tissue section. The sample tube was heated at 95° C. for 90 min., vortexing every 30 min. during the incubation. The sample tube was centrifuged to spin down the liquid and then cooled on ice for 2 min. The sample tube was incubated at 37° C. for 1 hour, vortexing every 20 min. during the incubation. The sample tube was centrifuged to spin down the liquid and heated at 95° C. for 5 min. The sample tube was centrifuged for 10 min. at 15,000×g at 4° C. The soluble fraction was then recovered.

### C. The Qproteome FFPE Tissue Extraction System (QIAGEN, Valencia Calif., USA)

2 slides of 10 μm tissue sections from the same patient are used. The slides are immersed in xylene for 5 min.; this step is repeated once in new xylene solution for 5 min. The slides were immersed in 100% ethanol for 5 min. This step was repeated. The slides were then immersed in 85% ethanol for 1 min., and in 70% ethanol for 1 min. The slides were immersed in water for 1 min. Excess water was removed and the sections transferred to a clean Eppendorf tube using a razor blade.

100 μl of extraction buffer from the manufacturer were added into one Eppendorf tube containing the tissue section. The sample tube was heated at 100° C. for 20 min. The sample tube was centrifuged to spin down the liquid. It was then incubated at 80° C. for 2 hours. The extract was vortexed every 30 min. during the incubation. It was then centrifuged for 10 min. at 15,000×g at 4° C. and the soluble fraction recovered.

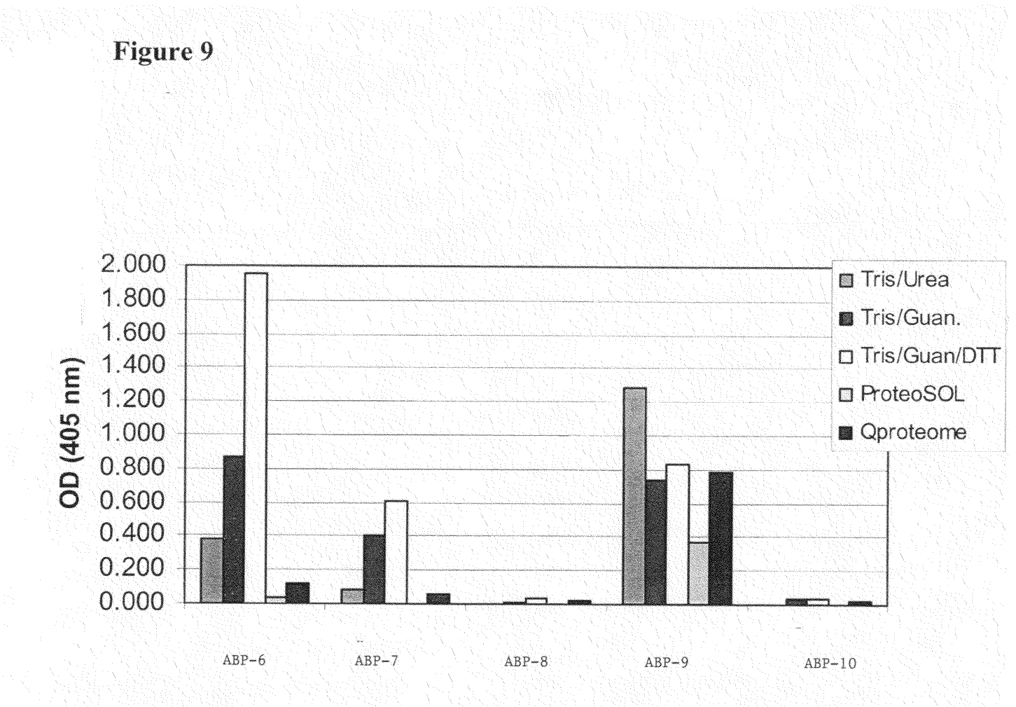

As can be seen in FIG. 9, the Tris/Guanidine/DTT extraction buffer provided by the invention is effective for extracting most tumor biomarkers from FFPE tissue sections. ABP-6 and ABP-7 were best extracted by the buffer of the invention.

### Example 4

### Protein Extraction from FFPE Tissue Sections From Breast Cancer Patients

FFPE tissue sections from patients were used (from USBiomax, Rockville, Md. and CYBRDI, Frederick Md.). The samples were from 20 malignant breast tumors and 19 benign breast tumors. The proteins were extracted form deparaffinized FFPE slides with the extraction buffer of the invention (20 mM Tris-HCl, pH 8.4, 6M GnHCl, 100 mM DTT, and protease inhibitors, which were 10 μM Leupeptin, 100 μM AEBSF, 0.3 μM Aprotinin, 1 μM Pepstatin A, 1 μM E-64 and 1 mM EDTA), by the method used in Example 3. The extracted proteins were quantified using a Bradford assay (Sigma-Aldrich, St. Louis, Mo.). ELISA was performed using amount of proteins per sample (1 μg proteins/well).

### EQUIVALENTS

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A protein extraction buffer comprising:
(a) 10 mM to 250 mM Tris-HCl, pH 7.0 to 10;
(b) 2 M to 8 M guanidine hydrochloride;
(c) 10 mM to 200 mM dithiothreitol; and
(d) a protease inhibitor;
the buffer being detergent-free.

2. The extraction buffer according to claim 1, wherein the protease inhibitor is selected from Leupeptin, AEBSF, Aprotinin, Pepstatin A, E-64, EDTA, and combinations thereof.

3. The protein extraction buffer according to claim 1, wherein the buffer comprises:
(a) 10 mM to 80 mM Tris-HCl, pH 8.0 to 8.6;
(b) 5 M to 8 M guanidine hydrochloride;
(c) 50 mM to 110 mM dithiothreitol; and
(d) a protease inhibitor.

4. The protein extraction buffer according to claim 1, comprising:
(a) 20 mM Tris-HCl, pH 8.4;
(b) 6 M guanidine hydrochloride;
(c) 100 mM dithiothreitol; and
(d) a protease inhibitor.

5. The protein extraction buffer according to claim 1, consisting essentially of:
(a) 20 mM Tris-HCl, pH 8.4;
(b) 6 M guanidine hydrochloride;
(c) 100 mM dithiothreitol; and
(d) a protease inhibitor.

6. A method for extracting protein from a formalin-fixed, paraffin-embedded tissue section comprising the steps of:
(a) obtaining a deparaffinized, formalin-fixed, paraffin-embedded tissue section;
(b) incubating the tissue section with a detergent-free protein extraction buffer comprising:
(i) 10 mM to 250 mM Tris-HCl, pH 7.0 to 10;
(ii) 2 M-8 M guanidine hydrochloride;
(iii) 10 mM-200 mM dithiothreitol; and
(iv) a protease inhibitor,
wherein the extraction buffer significantly preserves protein epitopes for antibody recognition on the extracted protein; and
(c) recovering from the contacted tissue section a soluble fraction containing the extraction buffer and one or more proteins.

7. The method according to claim 6, wherein the extraction buffer comprises:
(i) 10 mM to 80 mM Tris-HCl, pH 8.0 to 8.6;
(ii) 5 M to 8 M guanidine hydrochloride;
(iii) 50 mM to 110 mM dithiothreitol; and
(iv) a protease inhibitor.

8. The method according to claim 6, wherein the extraction buffer comprises:
(i) 20 mM Tris-HCl, pH 8.4;
(ii) 6 M guanidine hydrochloride;
(iii) 100 mM dithiothreitol; and
(iv) a protease inhibitor.

9. The method according to claim 6, wherein the extraction buffer consists essentially of:
(i) 20 mM Tris-HCl, pH 8.4;
(ii) 6 M guanidine hydrochloride;
(iii) 100 mM dithiothreitol; and
(iv) a protease inhibitor.

10. The method according claim 6, wherein the protease inhibitor is selected from Leupeptin, Aprotinin, Pepstatin A, E-64, EDTA, and combinations thereof.

11. The method according to claim 6, wherein the tissue section is incubated in the protein extraction buffer at two different temperatures.

12. The method according to claim 11, wherein the two different temperatures are about 100° C. and about 60° C.

13. The method according to claim 12, wherein the tissue section is first incubated in the protein extraction buffer at about 100° C. and then at about 60° C.

14. A kit for extracting protein from a deparaffinized, formalin-fixed, paraffin-embedded tissue section, the kit comprising:
(a) a detergent-free protein extraction buffer comprising:
(i) 10 mM to 250 mM Tris-HCl, pH 7.0 to 10;
(ii) 2 M to 8 M guanidine hydrochloride;
(iii) 10 mM-200 mM dithiothreitol; and
(iv) a protease inhibitor,
wherein the extraction buffer significantly preserves protein epitopes for antibody recognition on the extracted protein; and
(b) a manual for use.

15. The kit according to claim 14, wherein the extraction buffer comprises:
(i) 10 mM to 80 mM Tris-HCl, pH 8.0 to 8.6;
(ii) 5 M to 8 M guanidine hydrochloride;
(iii) 50 mM to 110 mM dithiothreitol; and
(iv) a protease inhibitor.

16. The kit according to claim 14, wherein the extraction buffer comprises:
(i) 20 mM Tris-HCl, pH 8.4;
(ii) 6 M guanidine hydrochloride;
(iii) 100 mM dithiothreitol; and
(iv) a protease inhibitor.

17. The kit according to claim 14, wherein the extraction buffer consists essentially of:
(i) 20 mM Tris-HCl, pH 8.4;
(ii) 6 M guanidine hydrochloride;
(iii) 100 mM dithiothreitol; and
(iv) a protease inhibitor.

18. The kit according to claims 14, wherein the protease inhibitor is Leupeptin, AEBSF, Aprotinin, Pepstatin A, E-64, EDTA, and/or combinations thereof.

19. A kit for extracting protein from a deparaffinized formalin-fixed paraffin-embedded tissue section comprising:
(a) 10 mM to 250 mM Tris-HCl, pH 7.0 to 10;
(b) 2 M to 8 M guanidine hydrochloride;
(c) 10 mM to 200 mM dithiothreitol; and
(d) a protease inhibitor, components (a)-(d) being components of a protein extraction buffer; and
(e) a manual for use and for directing the mixing of the individual components of the protein extraction buffer.

20. The kit according to claim 19, comprising the following components:
(a) 10 mM to 80 mM Tris-HCl, pH 8.0 to pH 8.6;
(b) 5 M to 8 M guanidine hydrochloride;
(c) 50 mM to 110 mM dithiothreitol; and
(d) a protease inhibitor.

21. The kit according to claim 19, comprising the following components:
(a) 20 mM Tris-HCl, pH 8.4;
(b) 6 M guanidine hydrochloride;
(c) 100 mM dithiothreitol; and
(d) a protease inhibitor.

22. The kit according to claim 19, consisting essentially of the following components:
(a) 20 mM Tris-HCl, pH 8.4;
(b) 6 M guanidine hydrochloride;
(c) 100 mM dithiothreitol; and
(d) a protease inhibitor.

23. The kit according to claim 19, wherein the protease inhibitor is selected from Leupeptin, AEBSF, Aprotinin, Pepstatin A, E-64 EDTA, and combination thereof.
